(19) United States
(12) Patent Application Publication
(10) Pub. No.: US 2006/0134188 A1
(43) Pub. Date: Jun. 22, 2006

(54) Transdermal pharmaceutical preparation with a progesterone A-specific ligand (PRASL) as active ingredient

(76) Inventors: Hans-Peter Podhaisky, Jena (DE); Stefan Bracht, Jena (DE)

Correspondence Address: STRIKER, STRIKER & STENBY, 103 EAST NECK ROAD, HUNTINGTON, NY, 11743, US

(21) Appl. No.: 11/311,752

(22) Filed: Dec. 19, 2005

Related U.S. Application Data
(60) Provisional application No. 60/637,588, filed on Dec. 20, 2004.

(30) Foreign Application Priority Data
Dec. 20, 2004 (DE) 10 2004 062 182.9

(51) Int. Cl. A61K 9/70; A61K 31/165

(52) U.S. Cl. 424/449; 514/621

(57) ABSTRACT

A transdermal patch for hormone therapy and fertility control has a backing layer, an effective-ingredient-containing adhesive layer adhering to the backing layer and a removable protective film. The adhesive layer includes a progestagenic effective ingredient and an estrogen in an adhesive matrix based on a silicone polymer, a polyisobutylene polymer (PIB), a polyacrylate polymer or a styrene block copolymer with butadiene or isoprene (SBS or SIS). The transdermal patch contains from 0.1 to 10%, based on a total weight of the adhesive matrix, of a progestagenic effective ingredient of formula I: 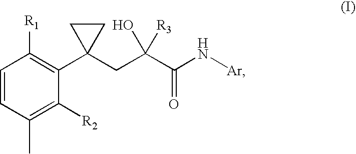
wherein R₁ and R₂ each represent, independently of each other, H or F;
R₃ represents CH₃ or CF₃ and
Ar is a group of formula II or III: 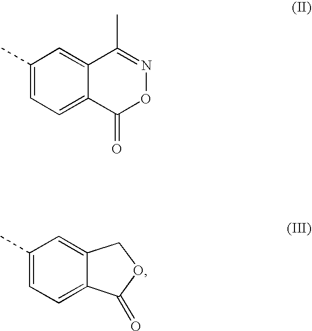 or a pharmaceutically suitable derivative thereof.

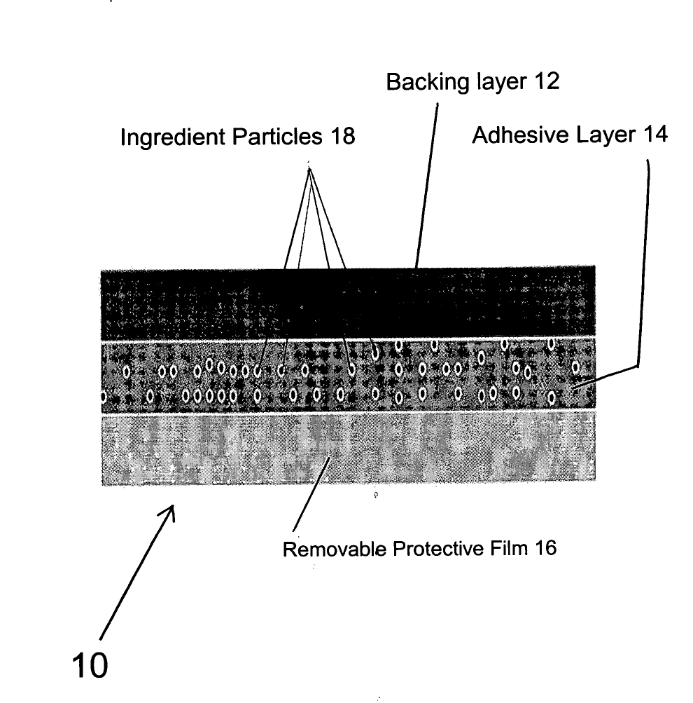

### CROSS-REFERENCE

U.S. Provisional Application No. 60/637,588, filed Dec. 20, 2004, and also DE 10 2004 062 182.9 disclose subject matter in common with that disclosed hereinbelow. Priority of invention is claimed on the basis of both the aforesaid U.S. Provisional Application and the DE application under 35 U.S.C. 119.

### BACKGROUND OF THE INVENTION

The subject matter of the present invention is a transdermal patch containing an effective ingredient having the following general formula I: 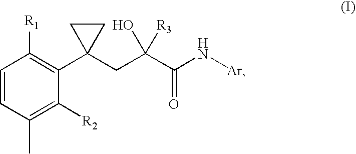
wherein R₁ and R₂ each represent, independently of each other, H or F;
R₃ represents CH₃ or CF₃ and
Ar is a group of formula II or III: 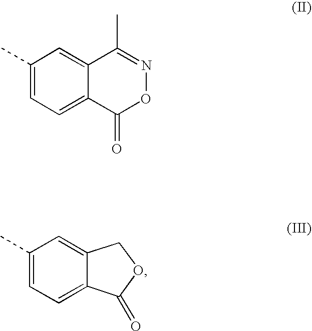
or a pharmaceutically suitable derivative thereof (progesterone A-specific ligand, PRASL);

wherein the transdermal patch comprises a backing layer, at least one effective ingredient-containing adhesive layer adhering to it and based on a silicone polymer, a polyisobutylene polymer (PIB), a polyacrylate polymer or a styrene block copolymer with butadiene or isoprene (SBS or SIS) and a removable protective film.

WO 03/075915 A1 from the patent literature discloses a transdermal application of PRASL. This reference describes a generally known transdermal patch, which optionally contains a penetration enhancer, or an emulsion salve, a cream or a gel.

Many known transdermal patches are passive matrix systems, comprising a largely moisture impermeable and effective-ingredient-impermeable backing layer, an effective ingredient containing adhesive layer and a removable protective layer. In currently known passive matrix systems the effective ingredient is completely dissolved in the adhesive layer.

However this form of preparation can have disadvantages.

The release rate of the dissolved effective ingredient from the patch matrix proceeds nonlinearly, but asymptotically approaches a maximum value, according to Fick's Second Law. This maximum value is, among other things, limited by the saturation concentration of the effective ingredient in the matrix. That means that the effective ingredient release rate from the patch per unit time and to the skin decreases with increasing application time. However a linear release of effective ingredient over the entire application time interval would be very desirable, especially for a hormonal medicinal substance, which should be taken over a long time interval of several days, since this corresponds very closely to the physiological secretion of hormones. Release kinetics of this sort is not attainable with the currently known transdermal patch.

The patent WO 03/075915 A1 proposes using an emulsion, a salve, a cream or a gel. The use of an emulsion, a salve, a cream or a gel for administering a PRASL-containing medicinal substance or drug appears to be generally unsuitable for several reasons. An exposed application of a high potency medicinal substance (PRASL activates pharmacological effects already with a daily dosage of 30 micrograms) must be considered problematical. Transdermal gels are generally applied over a large surface area of the skin (100 to 200 cm²). It is known that the major portion of the medicinal substance remains for a longer time on the skin surface and penetrates completely into the deeper skin layers in a time interval of several hours and then is reabsorbed. However because of that there is a considerably danger of partner contamination. A large amount of the effective ingredient can be transferred by contact with the skin of another individual and thus a non-participant can be exposed to an uncontrolled treatment. Furthermore part of the applied effective ingredient exposed on the skin surface can reach the shower drain during showering and thus cause environmental contamination.

Furthermore it is known that the transdermal availability of a medicinal substance is greatly limited by its molecular weight. Effective ingredients like PRASL with a molecular weight of greater than or equal to 500 Da are only slightly skin permeable. Because of that reason a higher amount or proportion of the effective ingredient PRASL must be used than in conventional patches, in order to obtain an effective plasma level of PRASL.

### SUMMARY OF THE INVENTION

It is an object of the present invention to provide a transdermal patch, which provides a pharmacologically effective plasma level of PRASL when about 30 to 50 micrograms/d are administered through the transdermal route.

In order to avoid the risk of effective ingredient-auxiliary substance incompatibilities and of irritating skin reactions, these patches should contain, in so far as it is possible, no penetration-amplifying auxiliary additive ingredients. Furthermore a transdermal patch with the effective ingredient PRASL should be available, which releases as large a portion of the working effective ingredient that is present as possible and, as a result, the patch contains as small a fraction of effective ingredient in its medicinal form as possible. In order to achieve an increase in acceptance by a patient by means of a reduction of the dosage interval, a transdermal patch of this sort with PRASL should be developed, which permits a linear effective transport over a time interval of several days.

According to the invention the transdermal patch contains an effective ingredient having the following general formula I: 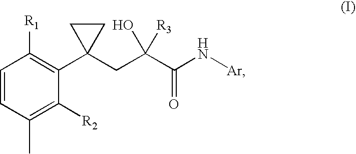
wherein R₁ and R₂ each represent, independently of each other, H or F;
R₃ represents CH₃ or CF₃ and
Ar is a group of formula II or III: 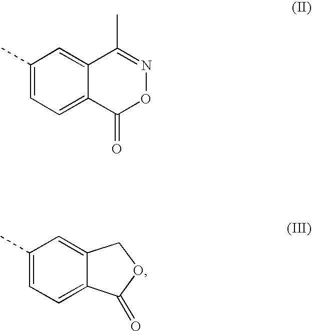
or a pharmaceutically suitable derivative thereof (progesterone A-specific ligand, PRASL);

wherein the transdermal patch comprises a backing layer, at least one effective ingredient-containing adhesive layer adhering to the backing layer and a removable protective film, and wherein the at least one adhesive layer comprises an effective ingredient and an adhesive matrix based on a silicone polymer, a polyisobutylene polymer (PIB), a polyacrylate polymer or a styrene block copolymer with butadiene or isoprene (SBS or SIS).

Furthermore the effective ingredient can be contained in a concentration of 0.1 to 10%, in relation to the total weight of the adhesive matrix, preferably in a concentration of from 0.1 to 5%, in relation to the total weight of the adhesive matrix. It is especially preferred when the effective ingredient concentration is in a range from 0.1 to 2% in the adhesive matrix.

Also in the transdermal patch according to the invention the solubility of the effective ingredient in the adhesive layer can be defined by 0.1 to 5%, preferably from 0.5 to 2%.

Less than 50% of the effective ingredient can be embedded in the matrix in undissolved form in the transdermal patch according to the invention.

The undissolved portion of the effective ingredient can be present as a uniform dispersion of microparticles or microdroplets, preferably as nanoparticles or nanodroplets. It is especially preferred that the effective ingredient is present in amorphous form.

The amorphous effective ingredient dispersion of the effective ingredient in the adhesive matrix provides the following clear advantages over the currently known crystalline dispersions:

the amorphous dispersion has an especially large boundary surface of the undissolved effective ingredient in the matrix, whereby the later dissolving of the effective ingredient for release from the matrix is simplified;

in the amorphous state no lattice energy must be overcome for dissolving of the effective ingredient during administration in contrast to crystalline effective ingredients, so that this dissolving process does not limit dispensing rate for release of the active ingredient from the matrix; and

the amorphous effective ingredient dispersion provides a uniform optical appearance; in a crystalline dispersion the user observes the occurrence of spots, which may suggest a reduced quality of the transdermal patch and thus produces an acceptance problem.

In the transdermal patch according to the present invention the crystallization inhibitor contained in the effective-ingredient-containing matrix can be selected from the group consisting of N-vinyl lactam polymers, such as N-vinyl-1-azacycloheptan-2-one homopolymers and N-vinyl-piperdin-2-one homopolymers and especially polymers of vinyl pyrrolidone, such as polyvidone (Collidone TM), or copolymers of vinyl pyrrolidones with vinyl acetate (copovidone).

The crystallization inhibitor can be a copovidone comprising 6 parts vinyl pyrrolidone and 4 parts vinyl acetate (Collidone TM VA 64).

The adhesive layer in the transdermal patch according to the invention comprises a silicone-based adhesive, which is characterized by a high proportion of polymer in comparison to resin, preferably an amine-compatible adhesive with a weight ratio of polymer to resin of greater than or equal to a limiting value in a range from 40% to 60%.

Furthermore the adhesive layer in the transdermal patch according to the invention can be an adhesive material based on polyisobutylene.

Also the transdermal patch can also contain an estrogen, which is selected from the group consisting of 17β-estradiol, ethinyl estradiol, estradiol valerate, estradiol cypionate, estradiol lactate and estradiol benzoate.

Furthermore more than 30%, preferably more than 50%, of the amount of the effective ingredient in the transdermal patch according to the invention can be released within a 7-day application period.

The transdermal patch can be made by a process in which the appropriate effective ingredient is taken up by a combination of at least two process solvents, one of which has a comparatively low solubility for the effective ingredient, while the other has a comparatively high solubility for the effective ingredient. The latter is removed from the batch after mixing with the adhesive matrix by a drying process.

The solvent with the low solubility for the effective ingredient can be 1, 4 dioxane. The solvent with the high solubility for the effective ingredient can be heptane.

In more detail according to the invention the manufacture of the PRASL patch is characterized by the following formulation strategy.

1. Selection of the adhesive matrix with optimum solvating properties for PRASL.

2. A suitable method of manufacture.

3. Selection of suitable stabilizing additives for the matrix.

I. Selection of the Adhesive Matrix

The selection of suitable adhesive matrices occurs based on the solubility of PRASL in the adhesive matrices. Suitable adhesive matrices for the present invention are those in which the solubility of PRASL is between 0.1 to 5%. Adhesive matrices, in which the solubility of PRASL is between 0.1 to 2%, have proven to be especially suitable.

For example, silicone, polyacrylate adhesives or polyisobutylene adhesives can be used as medicinally acceptable adhesives in these adhesive matrices. Moreover polyurethane, block copolymers based on styrene and additional organic polymers are usable.

Silicone adhesives, which are suitable for medicinal purposes and which have as great as possible a portion of the polymer in comparison to the resin, are particularly preferred in the transdermal patch according to the invention. Especially those silicone adhesives, which are amine compatible, such as the Bio PSA® series of Dow Corning and polyisobutylene-containing adhesive preparations, such as a preparation from the following ingredients, which are made in a known manner, are especially preferred.

For example, a polyisobutylene adhesive matrix for the transdermal patch according to the invention has the following composition.

| | Weight proportion in the |
|---|---|
| Ingredient | Dry Adhesive Matrix |
| PRASL | 1 |
| Oppanol B100 | 10 |
| Oppanol B 12 SFN | 52.2 |
| Indopol H 2100 | 35 |

2. Manufacturing Methods for Incorporating PRASL in the Matrix

During the manufacturing process PRASL is taken up in a combination of at least two process solvents, of which one solvent has a low solubility for the effective ingredient (e.g. heptane) and the other has a high solubility for the effective ingredient (e.g. 1,4 dioxane). One of the two solvents can also be a part of the volatile ingredients in the adhesive matrix. After mixing the PRASL solution with the adhesive matrix next the solvent, which has good solubility for the PRASL, is removed from the mixture by drying process.

Surprisingly a portion of the active ingredient in the form of an amorphous dispersion, whose particles are largely nanoparticles, spontaneously precipitates during the subsequent film-forming process. The precipitation of this amorphous dispersion presupposes the use of an adhesive matrix with the above-described solvent properties for PRASL.

If the solubility of the adhesive matrix used for PRASL is larger than 2 to 5%, as e.g. in the case of the acrylate adhesive matrix, DuroTak 387-2287, the entire effective ingredient amount dissolves after film-formation and the advantages of the amorphous effective ingredient dispersion according to the present invention cannot be employed.

3. Selection of Suitable Stabilizing Additives for the Matrix

Crystallization inhibitors can be used to stabilize this amorphous dispersion of the effective ingredient in the adhesive matrix. These ingredients are a matter of pharmaceutical auxiliary substances, which are complex formers and which are known to those skilled in the art and which form solid solutions with the effective ingredient, which increase the solubility limits for the effective ingredient and decrease the tendency of the effective ingredient to recrystallize after removal of a process solvent or lowering of the temperature. The addition of crystallization inhibitors stabilizes the amorphous dispersion of the effective ingredient in the adhesive matrix, since additional precipitation of the dissolved portion of the active ingredient is prevented and furthermore conversion of the amorphous particles into crystalline particles is prevented.

### BRIEF DESCRIPTION OF THE DRAWING

The objects, features and advantages of the invention will now be illustrated in more detail with the aid of the following detailed description and examples, with reference to the accompanying sole FIGURE, which is a diagrammatic cross-sectional view of one embodiment of a transdermal patch according to the present invention.

### DETAILED DESCRIPTION OF THE INVENTION

One embodiment of the transdermal patch according to the invention is shown in the sole FIGURE.

The transdermal patch 10 in this embodiment consists of a backing layer 12, which is Hostaphan RN MED® 15; an adhesive layer 14 comprising an adhesive matrix containing 1% PRASL; and a removable protective film or layer 16, which is Scotchpack 9742®. Particles 18 of effective ingredient are dispersed through out the adhesive matrix of layer 14.

### EXAMPLES

The following table I includes exemplary compositions of the adhesive layer in the transdermal patch according to the invention.

Example 4 is now explained in further detail hereinbelow.

A 10% solution of the effective ingredient, (R)-3-{1-[2-fluoro-5-(trifluoromethyl)-phenyl]-cyclopropyl}-2-hydroxy-N-(phthalid-5-yl)-2-(trifluoromethyl)-propanamide,

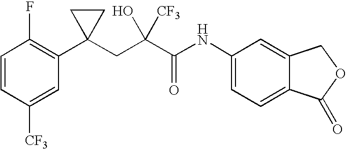

in dioxane is prepared in a suitable batch container. An aliquot of a suitable adhesive matrix (e.g. BioPSA® 4302, Dow Corning) is added, so that a 1% mixture, in relation to the solids content of the matrix, results.

TABLE I
ADHESIVE MATRIX COMPOSITIONS FOR TRANSDERMAL
PATCHES OF THE INVENTION

| | | Weight | |
|---|---|---|---|
| | | percent, | |
| Example | Effective Ingredient (R)/ | dry | |
| No. | adhesive matrix | ingredients | Comment |
| 1 | 3-{1-[2-fluoro-5-(trifluoroomethyl)- | 0.25 | An amorphous dis- |
| | phenyl]-cyclopropyl}-2-hydroxy- | | persion in the |
| | N-(phthalid-5-yl)-2-(trifluoromethyl)- | | adhesive matrix is not |
| | propanamide | | possible, since the |
| | | | effective ingredient is |
| | | | fully dissolved, even |
| | BioPSA ® 4302 | 99.75 | after film formation. |
| 2 | 3-{1-[2-fluoro-5-(trifluoromethyl)- | 0.5 | An amorphous dis- |
| | phenyl]-cyclopropyl}-2-hydroxy- | | persion in an |
| | N-(phthalid-5-yl)-2-(trifluoromethyl)- | | adhesive matrix is |
| | propanamide | | possible |
| | BioPSA ® 4302 | 99.5 | |
| 3 | 3-{1-[2-fluoro-5-(trifluoromethyl)- | 0.75 | An amorphous dis- |
| | phenyl]-cyclopropyl}-2-hydroxy- | | persion in an |
| | N-(phthalid-5-yl)-2-(trifluoromethyl)- | | adhesive matrix is |
| | propanamide | | possible |
| | BioPSA ® 4302 | 99.25 | |
| 4 | 3-{1-[2-fluoro-5-(trifluoromethyl)- | 1 | An amorphous dis- |
| | phenyl]-cyclopropyl}-2-hydroxy- | | persion in an |
| | N-(phthalid-5-yl)-2-(trifluoromethyl)- | | adhesive matrix is |
| | propanamide | | possible |
| | BioPSA ® 4302 | 99 | |
| 5 | 3-{1-[2-fluoro-5-(trifluoromethyl)- | 1 | An amorphous dis- |
| | phenyl]-cyclopropyl}-2-hydroxy- | | persion in an |
| | N-(phthalid-5-yl)-2-(trifluoromethyl)- | | adhesive matrix is |
| | propanamide | | possible |
| | Oppanol B100 | 10 | |
| | Oppanol B 12 SFN | 52.2 | |
| | Indopol H 2100 | 35 | |
| 6 | 3-{1-[2-fluoro-5-(trifluoromethyl)- | 1 | An amorphous dis- |
| | phenyl]-cyclopropyl}-2-hydroxy- | | persion in the |
| | N-(phthalid-5-yl)-2-(trifluoromethyl)- | | adhesive matrix is not |
| | propanamide | | possible, since the |
| | | | effective ingredient is |
| | | | fully dissolved, even |
| | DuroTak 387-2287 | 99 | after film formation. |
| 7 | 3-{1-[2-fluoro-5-(trifluoromethyl)- | 2 | An amorphous dis- |
| | phenyl]-cyclopropyl}-2-hydroxy- | | persion in the |
| | N-(phthalid-5-yl)-2-(trifluoromethyl)- | | adhesive matrix is not |
| | propanamide | | possible, since the |
| | | | effective ingredient is |
| | | | fully dissolved, even |
| | DuroTak 387-2287 | 98 | after film formation. |
| 8 | 3-{1-[2-fluoro-5-(trifluoromethyl)- | 3 | An amorphous dis- |
| | phenyl]-cyclopropyl}-2-hydroxy- | | persion in the |
| | N-(phthalid-5-yl)-2-(trifluoromethyl)- | | adhesive matrix is not |
| | propanamide | | possible, since the |
| | | | effective ingredient is |
| | | | fully dissolved, even |
| | DuroTak 387-2287 | 97 | after film formation. |
| 9 | 3-{1-[2-fluoro-5-(trifluoromethyl)- | 4 | An amorphous dis- |
| | phenyl]-cyclopropyl}-2-hydroxy- | | persion in the |
| | N-(phthalid-5-yl)-2-(trifluoromethyl)- | | adhesive matrix is not |
| | propanamide | | possible, since the |
| | | | effective ingredient is |
| | | | fully dissolved, even |
| | DuroTak 387-2287 | 96 | after film formation. |
| 10 | 3-{1-[2-fluoro-5-(trifluoromethyl)- | 5 | An amorphous dis- |
| | phenyl]-cyclopropyl}-2-hydroxy- | | persion in the |
| | N-(phthalid-5-yl)-2-(trifluoromethyl)- | | adhesive matrix is not |
| | propanamide | | possible, since the |
| | | | effective ingredient is |
| | DuroTak 387-2287 | 95 | fully dissolved, even |
| | | | after film formation. |

The matrix is homogenized in a known way. The adhesive layer is painted on are movable protective film (e.g. Scotchpack 9742®) and dried according to pharmaceutically standards. The effective ingredient is partially precipitated in the form of uniformly dispersed nanoparticles throughout the adhesive matrix. Subsequently coating with a largely moisture impermeable backing layer (e.g. Hostaphan RN 15 MED®, Misubishi) and separation of the finished transdermal patches occur.

One of the resulting patches is shown in the sole FIGURE and described above.

The following Table II reports the in vitro percentage release of the effective ingredient, (R) 3-{1-[2-fluoro-5-(trifluoromethyl)-phenyl]-cyclopropyl}-2-hydroxy-N-(phthalid-5-yl)-2-(trifluoromethyl)-propanamide, from six samples of the transdermal patch according to the invention containing 1% of the effective ingredient in the adhesive matrix, which is shown in the sole FIGURE.

TABLE II
PERCENTAGE RELEASE OF EFFECTIVE
INGREDIENT VS TIME

Time

| | 1 h | 4 h | 8 h | 24 h |
|---|---|---|---|---|
| Sample 1 | 39% | 72% | 80% | 81% |
| Sample 2 | 39% | 68% | 73% | 74% |
| Sample 3 | 40% | 74% | 91% | 92% |
| Sample 4 | 40% | 73% | 84% | 85% |
| Sample 5 | 39% | 73% | 85% | 87% |
| Sample 6 | 39% | 71% | 82% | 84% |
| Average | 39% | 72% | 83% | 84% |

Surprisingly the patch according to the invention releases considerably large portion of the effective ingredient for treatment after only four hours. The patch according to the invention is suitable to at least considerably reduce the overloading problem for oral medications comprising PRASL.

The transdermal patch according to the invention provides an economic advantage in comparison to an oral medication because of the reduction of the amount of the effective ingredient required in the administered form of the medication. Furthermore the reduction of the amount of the effective ingredient in each administered form considerably reduces the environmental risk, which accompanies the administered form.

The ability of the patch according to invention to transport the effective ingredient through the skin was tested by means of permeation studies with excised human skin.

For this purpose an established model of the Franz diffusion cell was used. Six sample patches were tested, which were each loaded with 1% of (R)-3-{1-[2-fluoro-5-(trifluoromethyl)-phenyl]-cyclopropyl}-2-hydroxy-N-(phthalid-5-yl)-2-(trifluoromethyl)-propanamide.

The Table III below shows the cumulative flux in micrograms per square cm of the effective ingredient through the excised human skin.

Surprisingly the results show that a maximum 15 cm² of transderamal patch according to the invention, which is free of penetration-enhancing auxiliary ingredients, is in a position to transport a therapeutically relevant amount (30 to 50 μg/d) of (R)-3-{1-[2-fluoro-5-(trifluoromethyl)-phenyl]-cyclopropyl}-2-hydroxy-N-(phthalid-5-yl)-2-(trifluoromethyl)-propanamide through the skin.

TABLE III
Cumulative Flux of 3-{1-[2-fluoro-5-(trifluoro-methyl)-phenyl]-
cyclopropyl}-2-hydroxy-N-(phthalid-5-yl)-2-(trifluoromethyl)-
propanamide through Excised Human Skin from a Patch Containing
1% of This Effective Ingredient

Time

| | 0 h | 6 h | 12 h | 24 h | 30 h | 48 h |
|---|---|---|---|---|---|---|
| Sample 1 | n.w. | n.w. | n.w. | 3.60 | 4.86 | 8.96 |
| Sample 2 | n.w. | n.w. | n.w. | 3.70 | 5.26 | 10.40 |
| Sample 3 | n.w. | n.w. | n.w. | 3.17 | 4.36 | 8.20 |
| Sample 4 | n.w. | n.w. | 0.92 | 5.45 | 7.03 | 11.96 |
| Sample 5 | n.w. | n.w. | n.w. | 3.35 | 4.78 | 9.51 |
| Sample 6 | n.w. | n.w. | n.w. | 2.94 | 3.97 | 7.33 |
| Average | n.w. | n.w. | n.w. | 3.70 | 5.04 | 9.39 |

n.w. = below detection limit.
Units of cumulate flux entered in Table III are μg/cm².

A high patient acceptance may be expected because of the reduced size of the transdermal patch. The risk of effective ingredient auxiliary agent incompatibilities and of irritating skin reactions is reduced by dispensing with penetration-enhancing additives. Thus the patch according to the present invention provides considerable advantages.

The administered form of the medication according to the invention permits a reduction of the dosage intervals from daily in the case of orally administered medications to weekly in the case of the transdermal patch of the invention because of the uniform flux rate for the effective ingredient.

These reductions provide additional advantages for the form of administration according to the present invention.

Unless otherwise indicated, all percentages are percentages by weight.

The disclosure in German Patent Application 10 2004 062 182.9 is incorporated here by reference. This German Patent Application describes the invention described hereinabove and claimed in the claims appended hereinbelow and provides the basis for a claim of priority for the instant invention under 35 U.S.C. 119.

While the invention has been illustrated and described as embodied in a transdermal pharmaceutical preparation with a progesterone A-specific ligand (prasl) as active ingredient, it is not intended to be limited to the details shown, since various modifications and changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

We claim:

1. A transdermal patch comprising a backing layer, at least one effective-ingredient-containing adhesive layer adhering to the backing layer and a removable protective film, wherein said at least one effective-ingredient-containing adhesive layer comprises an effective ingredient and an adhesive matrix based on a silicone polymer, a polyisobutylene polymer, a polyacrylate polymer or a styrene block copolymer with butadiene or isoprene;
wherein said effective ingredient is contained in a concentration of from 0.1 to 10%, based on a total weight of the adhesive matrix; and
wherein said effective ingredient is a compound of formula I: 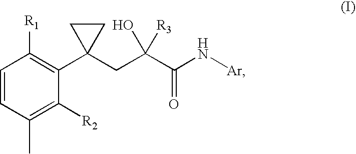
wherein R₁ and R₂ each represent, independently of each other, H or F;
R₃ represents CH₃ or CF₃ and
Ar is a group of formula II or III: 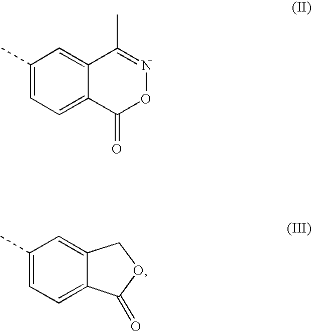
or a pharmaceutically suitable derivative thereof (progesterone A-specific ligand, PRASL).

2. The transdermal patch as defined in claim 1, wherein said concentration of said effective ingredient is from 0.1 to 5%, based on a total weight of the adhesive matrix.

3. The transdermal patch as defined in claim 1, wherein said concentration of said effective ingredient is from 0.1 to 2%, based on a total weight of the adhesive matrix.

4. The transdermal patch as defined in claim 1, wherein the effective ingredient has a solubility of 0.1 to 5% in said at least one effective-ingredient-containing adhesive layer.

5. The transdermal patch as defined in claim 1, wherein the effective ingredient has a solubility of 0.5 to 2% in said at least one effective-ingredient-containing adhesive layer.

6. The transdermal patch as defined in claim 1, wherein less than 50% of the effective ingredient is embedded in the adhesive matrix in undissolved form.

7. The transdermal patch as defined in claim 6, wherein the effective ingredient embedded in the adhesive matrix in undissolved form is in the form of microparticles and microdroplets dispersed in the adhesive matrix.

8. The transdermal patch as defined in claim 6, wherein the effective ingredient embedded in the adhesive matrix in undissolved form is in the form of nanoparticles and nanodroplets dispersed in the adhesive matrix.

9. The transdermal patch as defined in claim 6, wherein the effective ingredient embedded in the adhesive matrix in undissolved form is in an amorphous state.

10. The transdermal patch as defined in claim 1, wherein the at least one effective-ingredient-containing adhesive layer comprises a crystallization inhibitor and said crystallization inhibitor is selected from the group consisting of N-vinyl lactam polymers, polymers of vinyl pyrrolidone and copolymers of vinyl pyrrolidone and vinyl acetate.

11. The transdermal patch as defined in claim 1, wherein said adhesive martrix comprises a crystallization inhibitor and said crystallization inhibitor is selected from the group consisting of polyvidone, N-vinyl-1-aza-cycloheptan-2-one homopolymers and N-vinylpiperdin-2-one homopolymers.

12. The transdermal patch as defined in claim 1, wherein said adhesive matrix contains a copovidone as a crystallization inhibitor and said copovidone contains 6 parts vinyl pyrrolidone and 4 parts vinyl acetate.

13. The transdermal patch as defined in claim 1, wherein said at least one effective-ingredient-containing adhesive layer contains a silicone-based adhesive, and said silicone-based adhesive comprises a polymer and resin with a large portion of the polymer in comparison to the resin.

14. The transdermal patch as defined in claim 1, wherein said at least one effective-ingredient-containing adhesive layer contains a silicone-based adhesive, and wherein said silicone-based adhesive is amine-compatible and comprises a polymer and resin with a mass ratio of the polymer to the resin of greater than or equal to a value in a range between 40% and 60%.

15. The transdermal patch as defined in claim 1, wherein said at least one effective-ingredient-containing adhesive layer contains a polyisobutylene-based adhesive.

16. The transdermal patch as defined in claim 1, further comprising an estrogen selected from the group consisting of 17β-estradiol, ethinyl estradiol, estradiol valerate, estradiol cypionate, estradiol lactate and estradiol benzoate.

17. The transdermal patch as defined in claim 1, wherein more than 30% of a total amount of said effective ingredient contained therein is released during a seven day application cycle.

18. The transdermal patch as defined in claim 1, wherein more than 50% of a total amount of said effective ingredient contained therein is released during a seven day application cycle.

19. A method of making a transdermal patch,
wherein said transdermal patch comprises a backing layer, at least one effective-ingredient-containing adhesive layer adhering to the backing layer and a removable protective film, wherein said at least one effective-ingredient-containing adhesive layer comprises an effective ingredient and an adhesive matrix based on a silicone polymer, a polyisobutylene polymer, a polyacrylate polymer or a styrene block copolymer with butadiene or isoprene;
wherein said effective ingredient is contained in a concentration of from 0.1 to 10%, based on a total weight of the adhesive matrix; and
wherein said effective ingredient is a compound of formula I: 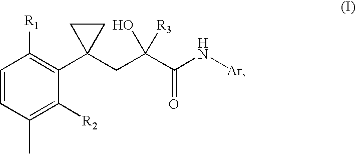
wherein R₁ and R₂ each represent, independently of each other, H or F;
R₃ represents CH₃ or CF₃ and
Ar is a group of formula II or III: 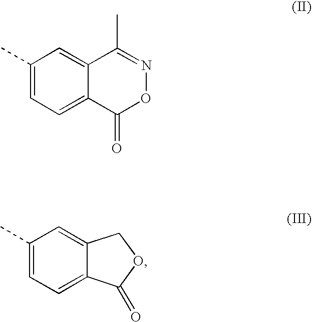
or a pharmaceutically suitable derivative thereof (progesterone A-specific ligand, PRASL); and
wherein said method comprises the steps of:
a) taking up the effective ingredient in a combination of solvents having a comparatively low solubility for the effective ingredient and a solvent having a comparatively high solubility for the effective ingredient to form an effective-ingredient-containing mixture;
b) mixing the effective-ingredient-containing mixture with the adhesive matrix to form a resulting batch; and
c) removing the solvent having the comparatively high solubility for the effective ingredient from the resulting batch.

20. The method as defined in claim 19, wherein the solvent with the comparatively low solubility for the effective ingredient is 1,4-dioxane and the solvent with the comparatively high solubility for the effective ingredient is heptane.
